(19) United States
(12) Plant Patent Application Publication
(10) Pub. No.: US 2007/0266467 P1
(43) Pub. Date: Nov. 15, 2007

(54) Anthurium plant named 'Barmodu'

(76) Inventor: Leonardus Johannes Maria Van Der Knaap, Naaldwijk (NL)

Correspondence Address: JONDLE & ASSOCIATES P.C., 858 HAPPY CANYON ROAD SUITE 230, CASTLE ROCK, CO, 80108, US

(21) Appl. No.: 11/432,643

(22) Filed: May 11, 2006

(51) Int. Cl. A01H 5/00

(52) U.S. Cl. PLT/263

(57) ABSTRACT

A new Anthurium plant particularly distinguished by 5 to 6 flowers with dark-pink, glimmering spathe, lilac-purple spadix, dark glimmering leaves that are narrowly below the flowers, and a compact growth habit, is disclosed.

### GENUS AND SPECIES

Anthurium andreanum.

### VARIETY DENOMINATION

‘Barmodu’.

### BACKGROUND OF THE NEW PLANT

The present invention comprises a new and distinct cultivar of Anthurium, botanically known as Anthurium andreanum, and hereinafter referred to by the cultivar name ‘Barmodu’. The new cultivar originated from a hybridization made in January 2000 in Maasland, The Netherlands. The female parent was the proprietary Anthurium plant ‘20040101-01’ (unpatented), while the male parent was the proprietary Anthurium plant ‘20040501-05’ (unpatented).

The new cultivar was selected from the results of the hybridization in 2001 and has been asexually reproduced repeatedly by tissue culture in Lochristi, Belgium and Maasland, The Netherlands over a five-year period. The present invention has been found to retain its distinctive characteristics through successive asexual propagations.

Plant Breeder's Rights for this cultivar were applied with the European Union on Nov. 15, 2005.

### SUMMARY OF THE INVENTION

The following are the most outstanding and distinguishing characteristics of this new cultivar when grown under normal horticultural practices in Maasland, The Netherlands.
1) 5 to 6 flowers;
2) Dark-pink glimmering spathe;
3) Lilac-purple spadix;
4) Flowers narrowly above dark, glimmering leaves; and
5) Compact growth habit.

### DESCRIPTION OF PHOTOGRAPHS

This new Anthurium plant is illustrated by the accompanying photographs which show the overall plant habit including blooms, buds and foliage of the plant; the colors shown are as true as can be reasonably obtained by conventional photographic procedures. The photographs are of a 56-week old plant grown in a greenhouse in Maasland, The Netherlands.

FIG. 1 shows the overall plant habit, including blooms, buds and foliage.

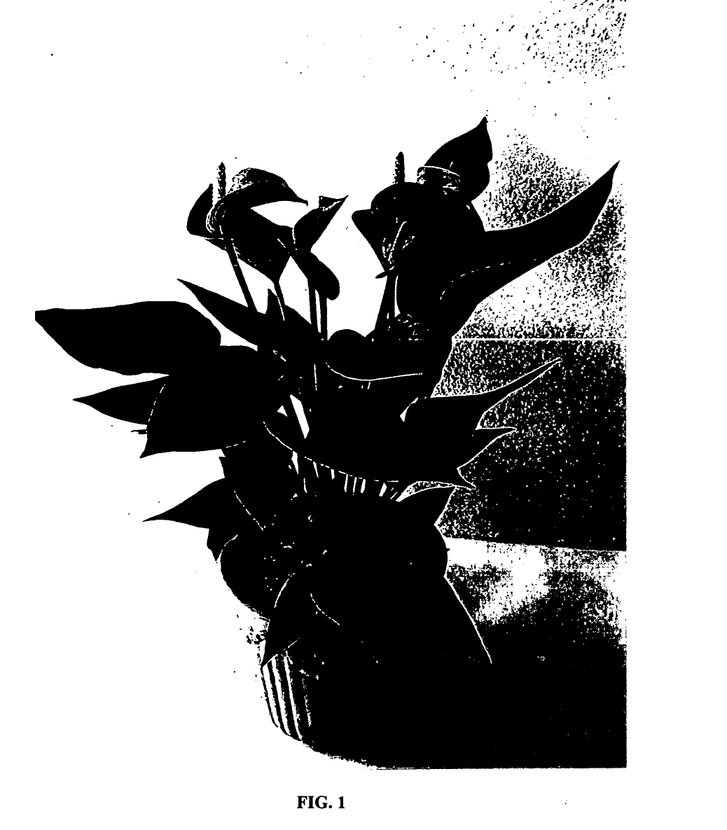

FIG. 2 shows the mature flower.

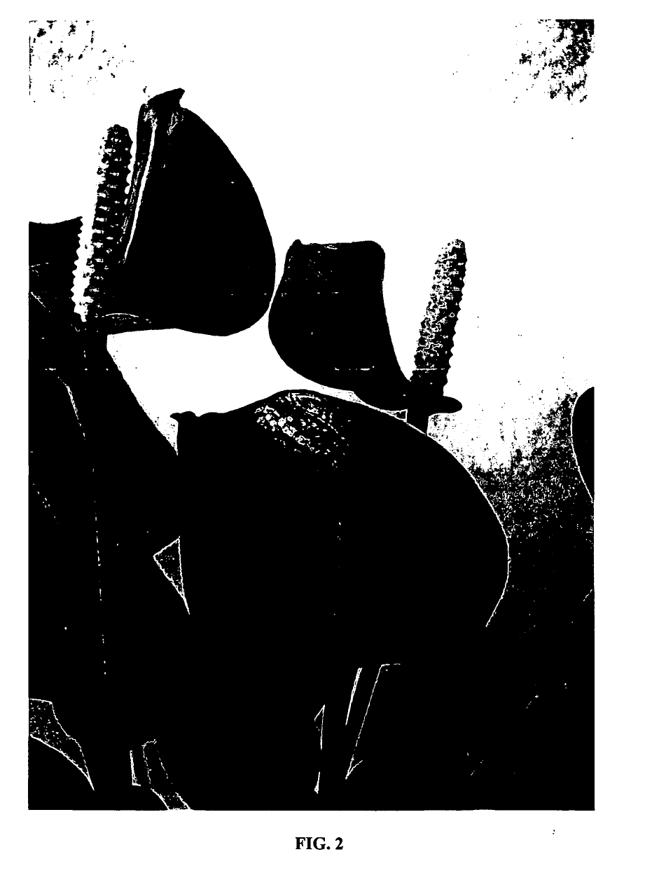

### DESCRIPTION OF THE NEW CULTIVAR

The following detailed description sets forth the distinctive characteristics of ‘Barmodu’. The data which define these characteristics were collected from asexual reproductions carried out in Maasland, The Netherlands. The plant history was taken on 56-week old plants which were planted from tissue culture in 14-cm pots and grown in a glass greenhouse between 19° C. and 24° C. Observations were made in the spring of 2006. Color readings were taken under 10 Klux in the greenhouse. Color references are primarily to the R.H.S. Colour Chart of The Royal Horticultural Society of London (R.H.S.) (2001).

### DETAILED BOTANICAL DESCRIPTION

Classification:
   Family.—Aracea.
   Botanical.—Anthurium andreanum.
   Common.—Anthurium.
Parentage:
   Female parent.—‘20040101-01’, a proprietary Anthurium plant (unpatented) with pink spathes.
   Male parent.—‘20040501-05’, a proprietary Anthurium plant (unpatented) with pink spathes and dark-purple spadix.
Propagation:
   Propagation.—Tissue culture.
   Time to initiate and develop roots.—10 days (under in vitro conditions).
   Root description.—RHS 159A to RHS 37B, freely branching, fine, fleshy with average density.
   Time to produce a finished flowering plant.—56 weeks for a 14 cm pot.
Plant:
   Form/shape.—Herbaceous perennial.
   Growth habit.—Upright. p2 Height (measured from soil, including inflorescence).—25 cm to 30 cm.
   Width (measured from leaf tips).—40 cm to 45 cm.
   Keeping quality.—90 days.
Leaves:
   Arrangement.—From the ground.
   Immature leaves.—Length: 6 cm to 8 cm Width: 4 cm to 5 cm Color: Upper surface: Darker than RHS 144A Lower surface: RHS 144A Texture: Smooth to leathery.
   Mature leaves.—Length (fully expanded): 15 cm to 17 cm Width: 10 cm Shape: Cordate Margin: Entire Apex: Apiculate to cuspidate Base: Subcordate Color: Upper surface: Closest to RHS 147A Lower surface: Closest to RHS 144A Texture: Leathery Venation: Pinnate Venation color: Upper surface: Closest to RHS 144A Lower surface: Closest to RHS 144B.
   Petiole.—Length: 5 cm to 20 cm Diameter: 0.2 cm to 0.3 cm Color: RHS 144A, oldest petioles overlain with anthocyan coloration Texture: Smooth, leathery.
   Geniculum.—Length: 1.5 cm to 2.0 cm Width: 0.2 cm to 0.3 cm Color: Upper surface: RHS N199A to RHS N199B Lower surface: Closest to RHS 144A Texture: Less smooth than petiole Wing: Length: Almost absent to 3 cm Width: 0.1 cm to 1.0 cm Color: RHS 144C to RHS 144D.
Inflorescence:
   Arrangement.—Single.
   Flowering habit (length of flowering season).—Continuous.
   Number of inflorescences per plant.—About 5 to 6.
   Fragrance.—Absent.
   Lastingness of individual blooms.—90 days.
Spathe:
   Shape.—Cordate.
   Apex.—Apiculate to cuspidate.
   Base.—Cordate.
   Margin.—Entire.
   Texture.—Leathery, somewhat blistered.
   Size.—Length: 6 cm to 7 cm Width: 4.5 cm to 6.0 cm.
   Color.—Immature: Front surface: Closest to RHS 54A Rear surface: Closest to RHS N66D Towards the base: RHS 54B to RHS 54C Mature: Front surface: Closest to RHS 52B Rear surface: Closest to RHS 63D Towards the base: RHS 54C, with RHS 147A dark veins.
Spadix:
   Size.—Length: 3.5 cm to 4.0 cm Diameter (at apex): About 0.6 cm Diameter (at base): About 0.7 cm
   Shape.—Straight, erect.
   Apex.—Rounded.
   Base.—Fused with spathe.
   Texture.—Shiny.
   Color.—Immature: RHS 39B at base to RHS 145B at apex Mature: Closest to RHS 159A.
Flowers:
   Quantity per spadix.—150 to 200.
   Shape.—Rounded.
   Size.—Length: 0.05 cm to 0.1 cm Diameter (max): 0.1 cm.
   Color.—RHS 159B.
Reproductive organs:
   Stamens.—Not observed.
   Pistil.—Quantity: 150 to 200 Length: Not observed.
   Style.—Not observed.
   Stigma.—Shape: Ovoid Diameter: About 0.05 cm Color: RHS 155B.
   Ovary.—Shape: Square Color: RHS 54D.
Fruit and seed set: Immature seed color: RHS 114B.
Disease and insect resistance: No particular resistance or susceptibility different from other Anthurium varieties.

### COMPARISON WITH PARENTAL AND COMMERCIAL CULTIVARS

‘Barmodu’ differs from the female parent ‘20040101-01’ (unpatented), by having dark-pink, lancet-shaped spathe, while ‘20040101-01’ has pink, more rounded-shaped spathe. In addition, ‘Barmodu’ has a compact plant habit and lancet-shaped leaves, while ‘20040101-01’ has a more stretched plant habit and Anthurium andreanum-shaped leaves.

‘Barmodu’ differs from the male parent ‘20040501-05’ (unpatented), by having dark-pink, large, lancet-shaped spathe and lilac-purple spadix, while ‘20040501-05’ has pink, medium, round-shaped spathe and dark-purple spadix. In addition, ‘Barmodu’ has medium to large leaves, while ‘20040501-05’ has small to medium leaves.

‘Barmodu’ differs from the commercial variety ‘Bossalo’ (unpatented), by having dark-pink spathe and lilac-purple spadix, while ‘Bossalo’ has red spathe and white-yellow spadix. In addition, ‘Barmodu’ has glimmering flowers and lancet-shaped glimmering leaves, while ‘Bossalo’ has dull flowers and Anthurium andreanum-shaped, dull leaves.

I claim:

1. A new and distinct cultivar of anthurium plant as shown and described herein.
